(19) United States
(12) Patent Application Publication
(10) Pub. No.: US 2019/0375766 A1
(43) Pub. Date: Dec. 12, 2019

(54) PROCESS FOR PREPARING HALOGENATED BICYCLIC SYSTEMS

(71) Applicant: Bayer CropScience Aktiengesellschaft, Monheim am Rhein (DE)

(72) Inventors: Marc MOSRIN, Koeln (DE); Ruediger FISCHER, Pulheim (DE); Dominik HAGER, Monheim (DE); Laura HOFFMEISTER, Duesseldorf (DE); Nina KAUSCH-BUSIES, Bergisch Gladbach (DE); David WILCKE, Duesseldorf (DE); Matthieu WILLOT, Duesseldorf (DE)

(21) Appl. No.: 16/331,843

(22) PCT Filed: Sep. 6, 2017
(86) PCT No.: PCT/EP2017/072363

§ 371 Date: Mar. 8, 2019

(30) Foreign Application Priority Data
Sep. 14, 2016 (EP) 16188760.9

(51) Int. Cl. C07F 3/06; C07D 471/04

(52) U.S. Cl. CPC C07F 3/06; C07D 471/04

(57) ABSTRACT

The present invention relates to a process for preparing halogenated bicyclic systems of the formula (II) proceeding from compounds of the structure Q-H via intermediates of the formula (IIIa) or (IIIb)

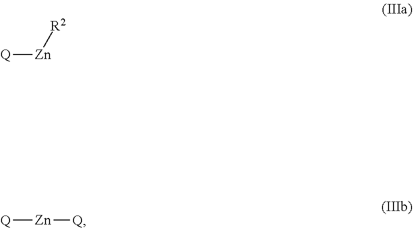

in which
Q is a structural element

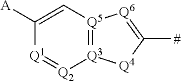

where the symbol # indicates the bond to the rest of the molecule and A, Q¹, Q², Q³, Q⁴, Q⁵ and Q⁶ have the definitions given in the description,
Hal is halogen, and
R² is halogen or —O-pivaloyl.

The present invention relates to a process for preparing halogenated bicyclic systems of the formula (II) Q-Hal (II), proceeding from compounds Q-H via intermediates of the formula (IIIa) or (IIIb)

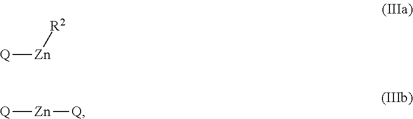

in which the structural elements shown in the formulae Q-H, (II), (IIIa) and (IIIb) have the definitions given below. The invention further relates to halogenated bicyclic systems and intermediates of this kind.

Halogenated bicyclic systems of the formula (II) are of great industrial significance for the pharmaceutical and agrochemical industry and are an important intermediate, inter alia, in the preparation of compounds that are effective as pesticides, for example.

The literature discloses that compounds of the formula (II) can be prepared by, in a first step, metallation in the presence of a very reactive lithium base, for example lithium diisopropylamide or n-butyllithium and subsequent reaction with a halide source, for example hexachloroethane or N-iodosuccinimide (as described in Journal of Organic Chemistry 2014, 79, 2203-2212 and Tetrahedron Letters 2012, 53, 1036-1041). It is likewise known that compounds of the formula (II) can be prepared by reaction of bicyclic hydroxyl compounds with phosphorus trichloride (as described in WO 2013/180193). However, the chemical synthesis methods that have been described in the prior art to date for such halogenated bicyclic systems very frequently make use of methods that are not economically implementable from an industrial point of view and/or have other disadvantages.

Disadvantages are the low chemical yields particularly in the case of highly substituted bicyclic systems, performance at very low temperatures for metallations (about −80° C.), and the generally difficult regio- and chemoselectivity of the halogenation. Furthermore, the introduction of bromine atoms or more particularly of iodine atoms into such bicyclic hydroxyl compounds is generally problematic or has not even been possible to date. Preparation—if possible at all—is therefore very costly and unsuitable for industrial scale commercial processes. Moreover, corresponding compounds are barely commercially available. This is especially true of 7-methyl-7H-imidazo[4,5-c]pyridazine, 3-methyl-3H-imidazo[4,5-c]pyridine and 3-methyl-3H-imidazo[4,5-b]pyridine.

With regard to the disadvantages outlined above, there is an urgent need for a simplified, industrially and economically performable process for preparing halogenated bicyclic systems, especially halogenated bicyclic systems of the formula (II). The halogenated bicyclic systems obtainable by this process sought are preferably to be obtained with good yield, high purity and in an economic manner.

It has been found that, surprisingly, halogenated bicyclic systems of the formula (II) can be prepared advantageously in a process using an organozinc base.

The present invention accordingly provides a process for preparing compounds of formula (II)

Q-Hal (II),

in which (configuration 1)

Q is a structural element

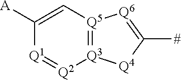

where the symbol # indicates the bond to the rest of the molecule and
Q¹ is N or CR⁶,
Q² is N or CR⁶,
Q³ is N or C,
Q⁴ is O, S, N or NR⁷,
Q⁵ is N or C,
Q⁶ is N or CH,
R⁶ is hydrogen, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
R⁷ is (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl, and
A is hydrogen, cyano, halogen, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, (C₁-C₄)alkoxy, (C₁-C₄)haloalkoxy, (C₁-C₄)alkoxyimino, (C₁-C₄)alkylthio, (C₁-C₄)haloalkylthio, (C₁-C₄)alkylsulphinyl, (C₁-C₄)haloalkylsulphinyl, (C₁-C₄)alkylsulphonyl, (C₁-C₄)haloalkylsulphonyl, (C₁-C₄)alkylsulphonyloxy, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, aminosulphonyl, (C₁-C₄)alkylaminosulphonyl or di-(C₁-C₄)alkylaminosulphonyl,
or A is —O—CF₂—O— and, together with Q¹ and the carbon atom to which it is bonded, forms a five-membered ring where Q¹ is carbon, and

Hal is halogen,

characterized in that, in a first process step a), a compound Q-H in which Q is as defined above

is reacted with an organozinc base of the structure (NR³R⁴)—Zn—R² or (NR³R⁴)₂—Zn in which

R² is halogen or —O-pivaloyl and

R³ and R⁴ together form a —(CH₂)₄—, —(CH₂)₅— or —(CH₂)₂O(CH₂)₂— group, where each of these groups may optionally be substituted by 1, 2, 3 or 4 R⁵ radicals and R⁵ is selected from the group consisting of methyl, ethyl, n-propyl and i-propyl,

to give a compound of the formula (IIIa) or the formula (IIIb)

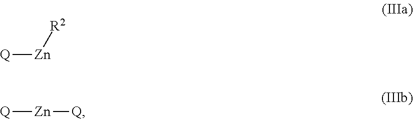

in which Q and R² each have the definitions given above,

and this compound of the formula (IIIa) or (IIIb) is reacted in a second process step b) with a compound of the structure X-Hal in which X is halogen and Hal has the abovementioned definition to give the compound of the formula (II).

The compound X-Hal, as apparent from the definitions of X and Hal, is an interhalogen compound, preferably elemental halogen.

Preferably, Q¹, Q², Q³, Q⁴, Q⁵ and Q⁶ represent not more than five nitrogen atoms overall and further preferably not more than four nitrogen atoms overall.

Preferred and particularly preferred definitions of the Q, X, Hal and R² radicals included in the aforementioned formulae Q-H, (II), (IIIa) and (IIIb) of the process of the invention are elucidated hereinafter, with more specific description of the organozinc base further down, and so the preferred configurations of the base are specified at that point.

### (Configuration 2)

Q is preferably a structural element from the group of Q1 to Q15

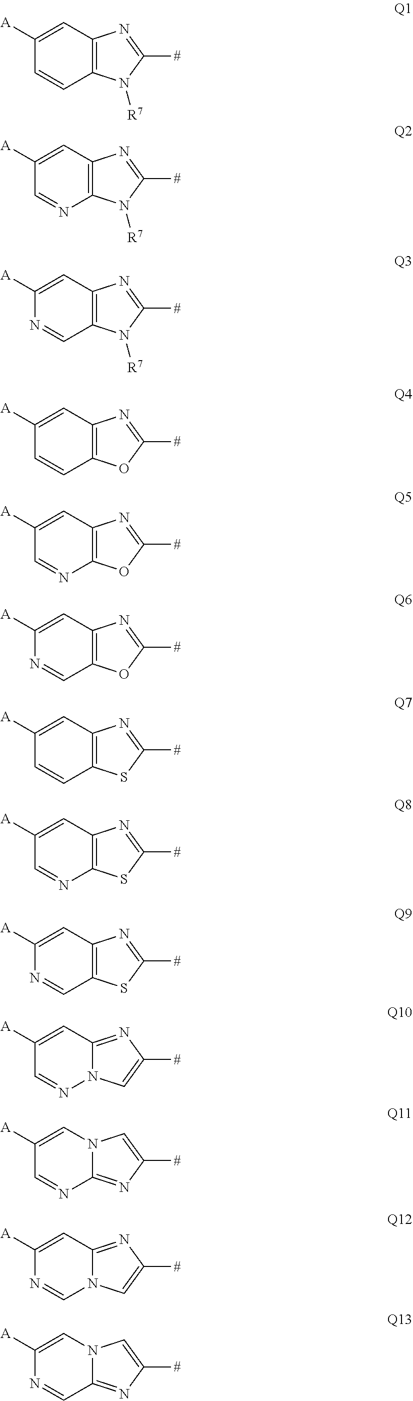

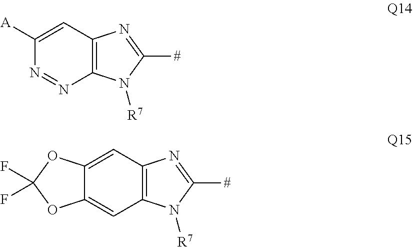

R⁷ is preferably (C₁-C₄)alkyl, (C₁-C₄)haloalkyl, (C₁-C₄)alkoxy-(C₁-C₄)alkyl, (C₁-C₄)haloalkoxy-(C₁-C₄)alkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
A is preferably fluorine, chlorine, bromine, fluoromethyl, difluoromethyl, trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethoxy, difluorochloromethoxy, dichlorofluoromethoxy, trifluoromethylthio, trifluoromethylsulphinyl or trifluoromethylsulphonyl,
Hal and X have the same definition and are preferably fluorine, chlorine, iodine or bromine, and
R² is preferably halogen, especially chlorine, bromine or iodine,

### (Configuration 3)

Q is more preferably a structural element from the group of Q2, Q3, Q10, Q12, Q14 or Q15,
R⁷ is more preferably (C₁-C₄)alkyl or (C₁-C₄)alkoxy-(C₁-C₄)alkyl,
A is more preferably trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethylthio, trifluoromethylsulphinyl or trifluoromethylsulphonyl,
Hal and X have the same definition and are more preferably iodine or bromine, and
R² is more preferably chlorine,

### (Configuration 4)

Q is most preferably the structural element Q2, Q3, Q12 or Q14,

R⁷ is most preferably methyl, ethyl, n-propyl or isopropyl, especially methyl,

A is most preferably trifluoromethyl,

Hal and X have the same definition and are most preferably iodine or bromine, and

R² is most preferably chlorine.

The radical definitions and elucidations given above apply both to the end products and intermediates and to the starting materials in a corresponding manner. These radical definitions can be combined with one another as desired, i.e. including combinations between the respective ranges of preference.

Preference is given in accordance with the invention to those compounds in which there is a combination of the definitions listed above as being preferred.

Particular preference is given in accordance with the invention to those compounds in which there is a combination of the definitions listed above as being more preferred.

Very particular preference is given in accordance with the invention to those compounds in which there is a combination of the definitions listed above as being most preferred.

In a further preferred embodiment of the invention, Q is Q1 and R⁷, A, Hal, X and R² have the definitions given in configuration 1 or those given in configuration 2 or those given in configuration 3 or those given in configuration 4 (configuration 5).

In a further preferred embodiment of the invention, Q is Q2 and R⁷, A, Hal, X and R² have the definitions given in configuration 1 or those given in configuration 2 or those given in configuration 3 or those given in configuration 4 (configuration 6).

In a further preferred embodiment of the invention, Q is Q3 and R⁷, A, Hal, X and R² have the definitions given in configuration 1 or those given in configuration 2 or those given in configuration 3 or those given in configuration 4 (configuration 7).

In a further preferred embodiment of the invention, Q is Q4 and R⁷, A, Hal, X and R² have the definitions given in configuration 1 or those given in configuration 2 or those given in configuration 3 or those given in configuration 4 (configuration 8).

In a further preferred embodiment of the invention, Q is Q5 and R⁷, A, Hal, X and R² have the definitions given in configuration 1 or those given in configuration 2 or those given in configuration 3 or those given in configuration 4 (configuration 9).

In a further preferred embodiment of the invention, Q is Q6 and R⁷, A, Hal, X and R² have the definitions given in configuration 1 or those given in configuration 2 or those given in configuration 3 or those given in configuration 4 (configuration 10).

In a further preferred embodiment of the invention, Q is Q7 and R⁷, A, Hal, X and R² have the definitions given in configuration 1 or those given in configuration 2 or those given in configuration 3 or those given in configuration 4 (configuration 11).

In a further preferred embodiment of the invention, Q is Q8 and R⁷, A, Hal, X and R² have the definitions given in configuration 1 or those given in configuration 2 or those given in configuration 3 or those given in configuration 4 (configuration 12).

In a further preferred embodiment of the invention, Q is Q9 and R⁷, A, Hal, X and R² have the definitions given in configuration 1 or those given in configuration 2 or those given in configuration 3 or those given in configuration 4 (configuration 13).

In a further preferred embodiment of the invention, Q is Q10 and R⁷, A, Hal, X and R² have the definitions given in configuration 1 or those given in configuration 2 or those given in configuration 3 or those given in configuration 4 (configuration 14).

In a further preferred embodiment of the invention, Q is Q11 and R⁷, A, Hal, X and R² have the definitions given in configuration 1 or those given in configuration 2 or those given in configuration 3 or those given in configuration 4 (configuration 15).

In a further preferred embodiment of the invention, Q is Q12 and R⁷, A, Hal, X and R² have the definitions given in configuration 1 or those given in configuration 2 or those given in configuration 3 or those given in configuration 4 (configuration 16).

In a further preferred embodiment of the invention, Q is Q13 and R⁷, A, Hal, X and R² have the definitions given in configuration 1 or those given in configuration 2 or those given in configuration 3 or those given in configuration 4 (configuration 17).

In a further preferred embodiment of the invention, Q is Q14 and R⁷, A, Hal, X and R² have the definitions given in configuration 1 or those given in configuration 2 or those given in configuration 3 or those given in configuration 4 (configuration 18).

In a further preferred embodiment of the invention, Q is Q15 and R⁷, A, Hal, X and R² have the definitions given in configuration 1 or those given in configuration 2 or those given in configuration 3 or those given in configuration 4 (configuration 19).

Advantageously, the halogenated bicyclic systems of the formula (II) can be prepared by the process according to the invention with good yields and in high purity. A great advantage of the process according to the invention is its regioselectivity and the comparatively mild reaction conditions under which it can be conducted, essentially a result of its performability at distinctly higher temperatures compared to −80° C. The possibility of being able to introduce halogens at distinctly higher temperatures as well is very attractive, and processes according to the invention, even at such higher temperatures, tolerate functional groups such as trifluoromethyl or other electron-withdrawing groups that activate ortho positions without impairment of the existing regioselectivity. Moreover, because of the very good functional group tolerance of zinc reagents, zinc bases are very attractive. Overall, it is thus possible to prepare compounds of the formula (II) within a short time and in very good yields.

The process according to the invention can be elucidated by the following scheme (I):

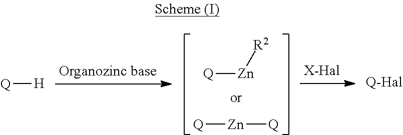

In this scheme, Q, X, Hal and R² and, within the respective definitions, any further structural elements present each have the definitions given above. The compounds shown in brackets are the intermediate (formula IIIa or formula IIIb) which are reacted further with a compound X-Hal to give the compound of the formula (II). Accordingly, the process according to the invention can be divided into the two process steps a) and b), step a) being the conversion of the compound Q-H to the respective intermediate and step b) being the further conversion of the intermediate to the compound of the formula (II).

### General Definitions

In the context of the present invention, the term halogen (Hal), unless defined otherwise, encompasses those elements selected from the group consisting of fluorine, chlorine, bromine and iodine.

The term “halides” in connection with the present invention describes compounds between halogens and elements of other groups of the Periodic Table, which can give rise to halide salts (ionic compounds (salts) which consist of anions and cations because of the great difference in electronegativity between the elements involved and are held together by electrostatic interactions) or covalent halides (covalent compounds where the difference in electronegativity is not as great as in the aforementioned ionic compounds, but the bonds have charge polarity), depending on the nature of the chemical bond. Particular preference is given in accordance with the invention to halide salts.

The term “pivaloyl” in the context of the present invention describes the deprotonated radical of pivalic acid (X) having the empirical formula (CH₃)₃CCO₂H.

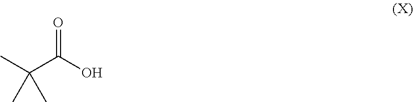

“O-pivaloyl” correspondingly means that the bond of the pivaloyl radical is via the deprotonated oxygen atom of the acid group.

In the context of the present invention, unless defined differently elsewhere, the term “alkyl”, either on its own or else in combination with further terms, for example haloalkyl, is understood to mean a radical of a saturated aliphatic hydrocarbon group which has 1 to 12 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl. Among these alkyl radicals, particular preference is given to C₁-C₆-alkyl radicals. Special preference is given to C₁-C₄-alkyl radicals.

According to the invention, unless defined differently elsewhere, the term “alkenyl”, either on its own or else in combination with further terms, is understood to mean a straight-chain or branched C₂-C₁₂-alkenyl radical which has at least one double bond, for example vinyl, allyl, 1-propenyl, isopropenyl, 1-butenyl, 2-butenyl, 3-butenyl, 1,3-butadienyl, 1-pentenyl, 2-pentenyl, 3-pentenyl, 4-pentenyl, 1,3-pentadienyl, 1-hexenyl, 2-hexenyl, 3-hexenyl, 4-hexenyl, 5-hexenyl and 1,4-hexadienyl. Among these, preference is given to C₂-C₆-alkenyl radicals and particular preference to C₂-C₄-alkenyl radicals.

According to the invention, unless defined differently elsewhere, the term “alkynyl”, either on its own or else in combination with further terms, is understood to mean a straight-chain or branched C₂-C₁₂-alkynyl radical which has at least one triple bond, for example ethynyl, 1-propynyl and propargyl. Among these, preference is given to C₃-C₆-alkynyl radicals and particular preference to C₃-C₄-alkynyl radicals. The alkynyl radical may also contain at least one double bond.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl. Among these, preference is given to C₃-C₆-cycloalkyl radicals.

The term “alkoxy”, either on its own or else in combination with further terms, for example haloalkoxy, is understood in the present case to mean an O-alkyl radical, where the term “alkyl” is as defined above.

Halogen-substituted radicals, for example haloalkyl, are mono- or polyhalogenated, up to the maximum number of possible substituents. In the case of polyhalogenation, the halogen atoms may be identical or different. Unless defined differently, halogen here is fluorine, chlorine, bromine or iodine, especially fluorine, chlorine or bromine. Alkyl groups substituted by one or more halogen atoms (-Hal) are, for example, selected from trifluoromethyl (CF₃), difluoromethyl (CHF₂), CF₃CH₂, ClCH₂ or CF₃CCl₂.

Unless stated otherwise, optionally substituted radicals may be mono- or polysubstituted, where the substituents in the case of poly substitutions may be the same or different.

The synthesis of compounds Q-H as reactants of a process according to the invention is known in principle to those skilled in the art. For example, compounds Q-H with Q=Q1, Q2, Q3, Q14 or Q15 can be obtained from corresponding pyridinediamine derivatives by ring closure to give the respective azole compound, as described, for example, in WO2014/100065 or WO2015/017610 preferably under acidic conditions. Alternative syntheses are likewise possible, but are more complex and as a result generally less economically advantageous.

The conversion of the compounds Q-H to compounds of the formula (IIIa) or (IIIb) in the first process step (step a)) is effected in the presence of an organozinc base of the structure (NR³R⁴)—Zn—R² or (NR³R⁴)₂—Zn, in which (configuration B-1)

R² is as defined above (configuration 1) (and is therefore halogen or —O-pivaloyl),

R³ and R⁴ together form a —(CH₂)₄—, —(CH₂)₅— or —(CH₂)₂O(CH₂)₂— group, where each of these groups may optionally be substituted by 1, 2, 3 or 4 R⁵ radicals and

R⁵ is selected from the group consisting of methyl, ethyl, n-propyl and i-propyl.

It is preferable that (configuration B-2)

R² is as defined above as preferred (configuration 2) (and is therefore halogen, especially chlorine, bromine or iodine),

R³ and R⁴ together form a —(CH₂)₅— group, where each of these groups may optionally be substituted by 1, 2, 3 or 4 R⁵ radicals and

R⁵ is selected from the group consisting of methyl and ethyl.

It is particularly preferable that (configuration B-3)

R² is as defined above as more preferred (configuration 3) or as most preferred (configuration 4) (and is therefore chlorine) and

R³ and R⁴ together form a —(CH₂)₅— group substituted by 4 methyl groups.

The radical definitions given above can be combined with one another as desired, i.e. including combinations between the respective ranges of preference.

In a very particularly preferred configuration of the base according to the invention, the structural element (NR³R⁴) is tetramethylpiperidine (TMP) of formula (IV).

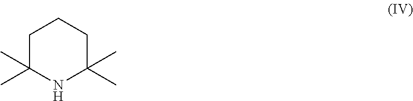

Organozinc bases most preferred in accordance with the invention are accordingly characterized in that zinc is bound to TMP, especially in the form of zinc halide and most preferably in the form of zinc chloride. Bases of this kind have the following structure of the formula (V) (configuration B-4)

(TMP)ₓZnCl₂₋ₓ, (V)

in which x is the number 1 or 2. Among these, preference is given in turn to bases with x=1 (configuration B-5) of formula (VI):

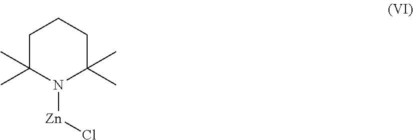

In a further preferred embodiment of the process according to the invention, the organozinc base is present in conjunction with alkali metal or alkaline earth metal halides. This is especially true of bases of the formulae (V) and (VI). Particularly preferred alkali metal or alkaline earth metal halides of this kind are lithium chloride and magnesium chloride, very particular preference being given to lithium chloride. Organozinc bases that are very particularly preferred in accordance with the invention are accordingly TMP ZnCl.LiCl or (TMP)₂ Zn.2LiCl (configuration B-6). Most preferred is TMP ZnCl.LiCl (VII; configuration B-7).

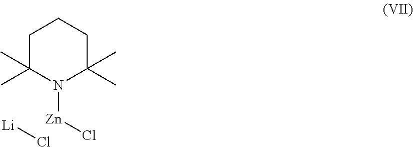

Specific combinations of compounds of the formulae Q-H, (II) and (IIIa) or (IIIb) with bases according to the invention are cited in table 1 below, these being employable in a process according to the invention. Since, in some configurations, the structural element R² is present both in the base according to the invention and in the compound of the formula (IIIa), the narrowest definition applies to R² in each case.

TABLE 1

| | Compounds of the formulae Q-H, (II) and | |
|---|---|---|
| Number | (IIIa) or (IIIb) | Base according to |
| 1 | Configuration 1 | Configuration B-1 |
| 2 | Configuration 1 | Configuration B-2 |
| 3 | Configuration 1 | Configuration B-3 |
| 4 | Configuration 1 | Configuration B-4 |
| 5 | Configuration 1 | Configuration B-5 |
| 6 | Configuration 1 | Configuration B-6 |
| 7 | Configuration 1 | Configuration B-7 |
| 8 | Configuration 2 | Configuration B-1 |
| 9 | Configuration 2 | Configuration B-2 |
| 10 | Configuration 2 | Configuration B-3 |
| 11 | Configuration 2 | Configuration B-4 |
| 12 | Configuration 2 | Configuration B-5 |
| 13 | Configuration 2 | Configuration B-6 |
| 14 | Configuration 2 | Configuration B-7 |
| 15 | Configuration 3 | Configuration B-1 |
| 16 | Configuration 3 | Configuration B-2 |
| 17 | Configuration 3 | Configuration B-3 |
| 18 | Configuration 3 | Configuration B-4 |
| 19 | Configuration 3 | Configuration B-5 |
| 20 | Configuration 3 | Configuration B-6 |
| 21 | Configuration 3 | Configuration B-7 |
| 22 | Configuration 4 | Configuration B-1 |
| 23 | Configuration 4 | Configuration B-2 |
| 24 | Configuration 4 | Configuration B-3 |
| 25 | Configuration 4 | Configuration B-4 |
| 26 | Configuration 4 | Configuration B-5 |
| 27 | Configuration 4 | Configuration B-6 |
| 28 | Configuration 4 | Configuration B-7 |
| 29 | Configuration 5 | Configuration B-1 |
| 30 | Configuration 5 | Configuration B-2 |
| 31 | Configuration 5 | Configuration B-3 |
| 32 | Configuration 5 | Configuration B-4 |
| 33 | Configuration 5 | Configuration B-5 |
| 34 | Configuration 5 | Configuration B-6 |
| 35 | Configuration 5 | Configuration B-7 |
| 36 | Configuration 6 | Configuration B-1 |
| 37 | Configuration 6 | Configuration B-2 |
| 38 | Configuration 6 | Configuration B-3 |
| 39 | Configuration 6 | Configuration B-4 |
| 40 | Configuration 6 | Configuration B-5 |
| 41 | Configuration 6 | Configuration B-6 |
| 42 | Configuration 6 | Configuration B-7 |
| 43 | Configuration 7 | Configuration B-1 |
| 44 | Configuration 7 | Configuration B-2 |
| 45 | Configuration 7 | Configuration B-3 |
| 46 | Configuration 7 | Configuration B-4 |
| 47 | Configuration 7 | Configuration B-5 |
| 48 | Configuration 7 | Configuration B-6 |
| 49 | Configuration 7 | Configuration B-7 |
| 50 | Configuration 8 | Configuration B-1 |
| 51 | Configuration 8 | Configuration B-2 |
| 52 | Configuration 8 | Configuration B-3 |
| 53 | Configuration 8 | Configuration B-4 |
| 54 | Configuration 8 | Configuration B-5 |
| 55 | Configuration 8 | Configuration B-6 |
| 56 | Configuration 8 | Configuration B-7 |
| 57 | Configuration 9 | Configuration B-1 |
| 58 | Configuration 9 | Configuration B-2 |
| 59 | Configuration 9 | Configuration B-3 |
| 60 | Configuration 9 | Configuration B-4 |
| 61 | Configuration 9 | Configuration B-5 |
| 62 | Configuration 9 | Configuration B-6 |
| 63 | Configuration 9 | Configuration B-7 |
| 64 | Configuration 10 | Configuration B-1 |
| 65 | Configuration 10 | Configuration B-2 |
| 66 | Configuration 10 | Configuration B-3 |
| 67 | Configuration 10 | Configuration B-4 |
| 68 | Configuration 10 | Configuration B-5 |
| 69 | Configuration 10 | Configuration B-6 |
| 70 | Configuration 10 | Configuration B-7 |
| 71 | Configuration 11 | Configuration B-1 |
| 72 | Configuration 11 | Configuration B-2 |
| 73 | Configuration 11 | Configuration B-3 |
| 74 | Configuration 11 | Configuration B-4 |
| 75 | Configuration 11 | Configuration B-5 |
| 76 | Configuration 11 | Configuration B-6 |
| 77 | Configuration 11 | Configuration B-7 |
| 78 | Configuration 12 | Configuration B-1 |
| 79 | Configuration 12 | Configuration B-2 |
| 80 | Configuration 12 | Configuration B-3 |
| 81 | Configuration 12 | Configuration B-4 |
| 82 | Configuration 12 | Configuration B-5 |
| 83 | Configuration 12 | Configuration B-6 |
| 84 | Configuration 12 | Configuration B-7 |
| 85 | Configuration 13 | Configuration B-1 |
| 86 | Configuration 13 | Configuration B-2 |
| 87 | Configuration 13 | Configuration B-3 |
| 88 | Configuration 13 | Configuration B-4 |
| 89 | Configuration 13 | Configuration B-5 |
| 90 | Configuration 13 | Configuration B-6 |
| 91 | Configuration 13 | Configuration B-7 |
| 92 | Configuration 14 | Configuration B-1 |
| 93 | Configuration 14 | Configuration B-2 |
| 94 | Configuration 14 | Configuration B-3 |
| 95 | Configuration 14 | Configuration B-4 |
| 96 | Configuration 14 | Configuration B-5 |
| 97 | Configuration 14 | Configuration B-6 |
| 98 | Configuration 14 | Configuration B-7 |
| 99 | Configuration 15 | Configuration B-1 |
| 100 | Configuration 15 | Configuration B-2 |
| 101 | Configuration 15 | Configuration B-3 |
| 102 | Configuration 15 | Configuration B-4 |
| 103 | Configuration 15 | Configuration B-5 |
| 104 | Configuration 15 | Configuration B-6 |
| 105 | Configuration 15 | Configuration B-7 |
| 106 | Configuration 16 | Configuration B-1 |
| 107 | Configuration 16 | Configuration B-2 |
| 108 | Configuration 16 | Configuration B-3 |
| 109 | Configuration 16 | Configuration B-4 |
| 110 | Configuration 16 | Configuration B-5 |
| 111 | Configuration 16 | Configuration B-6 |
| 112 | Configuration 16 | Configuration B-7 |
| 113 | Configuration 17 | Configuration B-1 |
| 114 | Configuration 17 | Configuration B-2 |
| 115 | Configuration 17 | Configuration B-3 |
| 116 | Configuration 17 | Configuration B-4 |
| 117 | Configuration 17 | Configuration B-5 |
| 118 | Configuration 17 | Configuration B-6 |
| 119 | Configuration 17 | Configuration B-7 |
| 120 | Configuration 18 | Configuration B-1 |
| 121 | Configuration 18 | Configuration B-2 |
| 122 | Configuration 18 | Configuration B-3 |
| 123 | Configuration 18 | Configuration B-4 |
| 124 | Configuration 18 | Configuration B-5 |
| 125 | Configuration 18 | Configuration B-6 |
| 126 | Configuration 18 | Configuration B-7 |
| 127 | Configuration 19 | Configuration B-1 |
| 128 | Configuration 19 | Configuration B-2 |
| 129 | Configuration 19 | Configuration B-3 |
| 130 | Configuration 19 | Configuration B-4 |
| 131 | Configuration 19 | Configuration B-5 |
| 132 | Configuration 19 | Configuration B-6 |
| 133 | Configuration 19 | Configuration B-7 |

Preferably, the organozinc base is used in the process according to the invention in a total amount of 0.5 to 5 equivalents, preferably of 0.8 to 2 equivalents, further preferably of 1 to 1.5 equivalents and more preferably of 1.0 to 1.2 equivalents, based on the compound Q-H. One advantage of the process according to the invention in this regard is that the organometallic base can be used in virtually stoichiometric amounts.

Depending on whether the structural element (NR³R⁴) is present once or twice in the organozinc base used, intermediate compounds of the formula (IIIa) or of the formula (IIIb) are formed in process step a).

The conversion of the compounds of the formula (IIIa) or (IIIb) to compounds of the formula (II) in the second process step (step b)) is effected in the presence of a compound X-Hal in which X and Hal each have the definitions given above.

Since both X and Hal are halogen, the compound is an interhalogen compound. X and Hal need not necessarily be the same halogen. For example, X may be iodine or bromine and Hal may be chlorine, bromine or iodine. Preferably, the compound X-Hal, however, is elemental halogen, especially F₂, Cl₂, Br₂ or I₂. Particular preference is given to I₂ or Br₂, very particular preference to 12.

Preferably, the compound X-Hal is used in the process according to the invention in a total amount of 0.5 to 10.0 equivalents, preferably of 0.8 to 5 equivalents, further preferably of 1 to 2.5 equivalents and more preferably of 1.0 to 2.0 equivalents, based on the compound Q-H.

The inventive conversion of the compounds Q-H to compounds of the formula (IIIa) or (IIIb) and further to compounds of the formula (II) is preferably effected in the presence of an organic solvent in each case. Useful solvents in principle include all organic solvents which are inert under the reaction conditions employed and in which the compounds to be converted have adequate solubility. Suitable solvents especially include: tetrahydrofuran (THF), 1,4-dioxane, diethyl ether, diglyme, methyl tert-butyl ether (MTBE), tert-amyl methyl ether (TAME), 2-methyl-THF, toluene, xylenes, mesitylene, ethylene carbonate, propylene carbonate, N,N-dimethylacetamide, N,N-dimethylformamide (DMF), N-methylpyrrolidone (NMP), N-ethyl-2-pyrrolidone (NEP), N-butyl-2-pyrrolidone (NBP); N,N′-dimethylpropyleneurea (DMPU), halohydrocarbons and aromatic hydrocarbons, especially chlorohydrocarbons such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, especially 1,2-dichlorobenzene, chlorotoluene, trichlorobenzene; 4-methoxybenzene, fluorinated aliphatics and aromatics, such as trichlorotrifluoroethane, benzotrifluoride and 4-chlorobenzotrifluoride. It is also possible to use solvent mixtures, preferably mixtures of the aforementioned solvents such as tetrahydrofuran (THF), 1,4-dioxane, diethyl ether, diglyme, methyl tert-butyl ether (MTBE), tert-amyl methyl ether (TAME), 2-methyl-THF, toluene, xylenes, mesitylene, dimethylformamide (DMF).

Preferred solvents are THF, N,N-dimethylformamide (DMF), 1,4-dioxane, diglyme, methyl tert-butyl ether (MTBE), tert-amyl methyl ether (TAME), 2-methyl-THF, toluene and 4-methoxybenzene.

Particularly preferred solvents are THF and N,N-dimethylformamide (DMF), very particular preference being given to THF.

The solvent may also be degassed (oxygen-free).

Preference is given to using the same solvent for both process steps a) and b). Alternative configurations of the invention in which different solvents are used for process steps a) and b) are likewise possible, however, in which case the solvents are likewise preferably selected from the aforementioned solvents, and the respective solvents specified as being preferred, more preferred and most preferred are applicable to the respective process step a) or b).

The conversion in process step a) is generally conducted at a temperature between 0° C. and 80° C. and with increasing preference between 10° C. and 70° C., between 15° C. and 60° C., between 20° C. and 50° C., between 20° C. and 40° C., and most preferably between 20° C. and 35° C., for example at room temperature or 25° C.

The conversion in process step b) is generally conducted at a temperature between 0° C. and 40° C. and with increasing preference between 0° C. and 35° C., between 0° C. and 30° C., and most preferably between 0° C. and 25° C., for example at room temperature or 25° C. It is particularly advantageous when reactions with elemental bromine (X and Hal are each bromine) are effected at 0° C. and reactions with elemental iodine (X and Hal are each iodine) at room temperature or 25° C.

The reaction is typically conducted at standard pressure, but can also be conducted at elevated or reduced pressure.

The desired compounds of the formula (II) can be isolated, for example, by aqueous workup in the presence of saturated ammonium chloride or sodium thiosulphate solutions and/or subsequent chromatography. Such processes are known to those skilled in the art and also include crystallization from an organic solvent or solvent mixture.

Two examples of particularly preferred embodiments of the process according to the invention can be elucidated with reference to the following schemes (IIa) and (IIb):

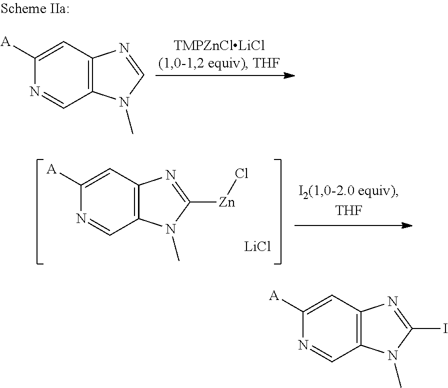

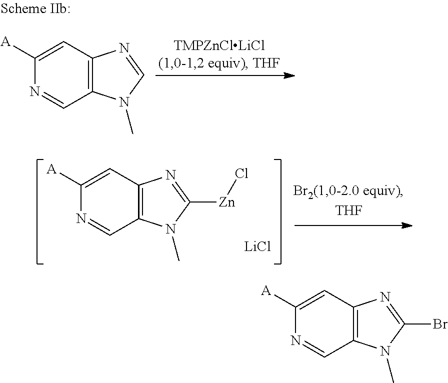

Scheme IIa and scheme IIb differ merely in that the reaction in process step b) is effected with elemental iodine (IIa) or with elemental bromine (IIb). In both schemes, A in each case has the definitions given above. The compound shown in brackets represents the corresponding intermediate of the formula IIIa which is converted further to the product, a compound of the formula (II). Both reactions take place in THF as solvent. “equiv” denotes the amount of equivalents of TMPZnCl.LiCl or compound X-Hal used, i.e. elemental iodine or elemental bromine here.

The present invention further provides compounds of the structure Q-H selected from the following compounds:

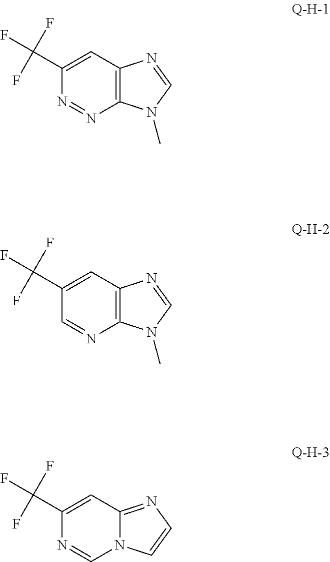

The present invention further provides compounds of the formula (IIIa) selected from the following compounds:

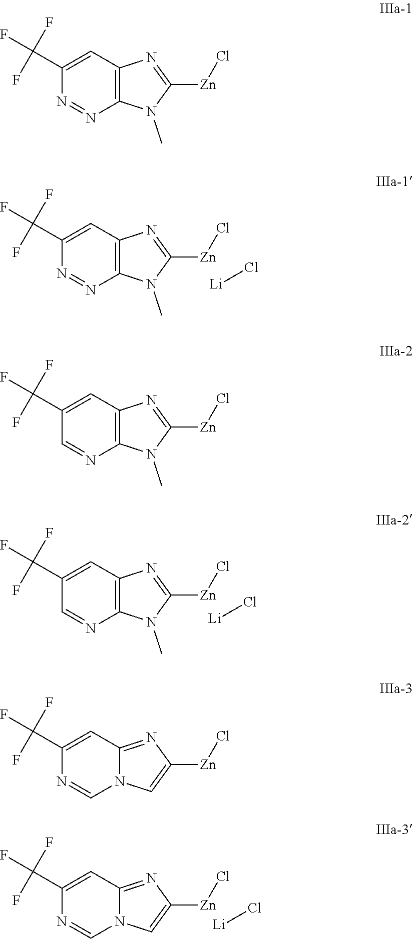

The present invention further provides compounds of the formula (II)

Q-Hal (II)

in which (configuration Q-Hal-1-1)

Q is a structural element

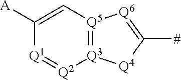

where the symbol # indicates the bond to the rest of the molecule and
Q¹ is N or CR⁶,
Q² is N or CR⁶,
Q³ is N or C,
Q⁴ is O, S, N or NR⁷,
Q⁵ is N or C,
Q⁶ is N or CH,
R⁶ is hydrogen, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,

R⁷ is (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,

A is hydrogen, cyano, halogen, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, (C₁-C₄)alkoxy, (C₁-C₄)haloalkoxy, (C₁-C₄)alkoxyimino, (C₁-C₄)alkylthio, (C₁-C₄)haloalkylthio, (C₁-C₄)alkylsulphinyl, (C₁-C₄)haloalkylsulphinyl, (C₁-C₄)alkylsulphonyl, (C₁-C₄)haloalkylsulphonyl, (C₁-C₄)alkylsulphonyloxy, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, aminosulphonyl, (C₁-C₄)alkylaminosulphonyl or di-(C₁-C₄)alkylaminosulphonyl,
or A is —O—CF₂—O— and, together with Q′ and the carbon atom to which it is bonded, forms a five-membered ring where Q′ is carbon, and

Hal is halogen.

Preferably, Q¹, Q², Q³, Q⁴, Q⁵ and Q⁶ represent not more than five nitrogen atoms overall and further preferably not more than four nitrogen atoms overall.

In an alternative configuration (configuration Q-Hal-1-2), the definitions of the radicals included in the aforementioned formula (II) are as follows:

Q is a structural element

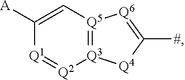

where the symbol # indicates the bond to the rest of the molecule and
Q¹ is N or CR⁶,
Q² is N or CR⁶,
Q³ is N or C,
Q⁴ is O, S, N or NR⁷,
Q⁵ is N or C,
Q⁶ is N or CH,
R⁶ is hydrogen, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
R⁷ is (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
A is hydrogen, cyano, halogen, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, (C₁-C₄)alkoxy, (C₁-C₄)haloalkoxy, (C₁-C₄)alkoxyimino, (C₁-C₄)alkylthio, (C₁-C₄)haloalkylthio, (C₁-C₄)alkylsulphinyl, (C₁-C₄)haloalkylsulphinyl, (C₁-C₄)alkylsulphonyl, (C₁-C₄)haloalkylsulphonyl, (C₁-C₄)alkylsulphonyloxy, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, aminosulphonyl, (C₁-C₄)alkylaminosulphonyl or di-(C₁-C₄)alkylaminosulphonyl,
or A is —O—CF₂—O— and, together with Q¹ and the carbon atom to which it is bonded, forms a five-membered ring where Q¹ is carbon, and

Hal is fluorine, iodine or bromine, especially iodine or bromine.

Preferred (configurations Q-Hal-2-1 and Q-Hal-2-2), particularly preferred (configuration Q-Hal-3-1) and very particularly preferred (configuration Q-Hal-4-1) definitions of the radicals of the compounds of the formula (II) that are included in the above configurations Q-Hal-1-1 and Q-Hal-1-2 are elucidated hereinafter.

### (Configuration Q-Hal-2-1)

Q is preferably a structural element from the group of Q1 to Q15,
R⁷ is preferably (C₁-C₄)alkyl, (C₁-C₄)haloalkyl, (C₁-C₄)alkoxy-(C₁-C₄)alkyl, (C₁-C₄)haloalkoxy-(C₁-C₄)alkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
A is preferably fluorine, chlorine, bromine, fluoromethyl, difluoromethyl, trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethoxy, difluorochloromethoxy, dichlorofluoromethoxy, trifluoromethylthio, trifluoromethylsulphinyl or trifluoromethylsulphonyl, and
Hal is preferably fluorine, chlorine, iodine or bromine.

### (Configuration Q-Hal-2-2)

Q is preferably a structural element from the group of Q1 to Q15,
R⁷ is preferably (C₁-C₄)alkyl, (C₁-C₄)haloalkyl, (C₁-C₄)alkoxy-(C₁-C₄)alkyl, (C₁-C₄)haloalkoxy-(C₁-C₄)alkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
A is preferably fluorine, chlorine, bromine, fluoromethyl, difluoromethyl, trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethoxy, difluorochloromethoxy, dichlorofluoromethoxy, trifluoromethylthio, trifluoromethylsulphinyl or trifluoromethylsulphonyl, and
Hal is preferably fluorine, iodine or bromine, especially iodine or bromine.

### (Configuration Q-Hal-3-1)

Q is more preferably a structural element from the group of Q2, Q3, Q10, Q12, Q14 or Q15,
R⁷ is more preferably (C₁-C₄)alkyl or (C₁-C₄)alkoxy-(C₁-C₄)alkyl,
A is more preferably trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethylthio, trifluoromethylsulphinyl or trifluoromethylsulphonyl, and
Hal is more preferably iodine or bromine.

### (Configuration Q-Hal-4-1)

Q is most preferably the structural element Q2, Q3, Q12 or Q14,

R⁷ is most preferably methyl, ethyl, n-propyl or isopropyl, especially methyl,

A is most preferably trifluoromethyl, and

Hal is most preferably iodine or bromine.

The radical definitions given above can be combined with one another as desired, i.e. including combinations between the respective ranges of preference.

Preference is given in accordance with the invention to those compounds in which there is a combination of the definitions listed above as being preferred.

Particular preference is given in accordance with the invention to those compounds in which there is a combination of the definitions listed above as being more preferred.

Very particular preference is given in accordance with the invention to those compounds in which there is a combination of the definitions listed above as being most preferred.

Examples of such particularly preferred compounds are:

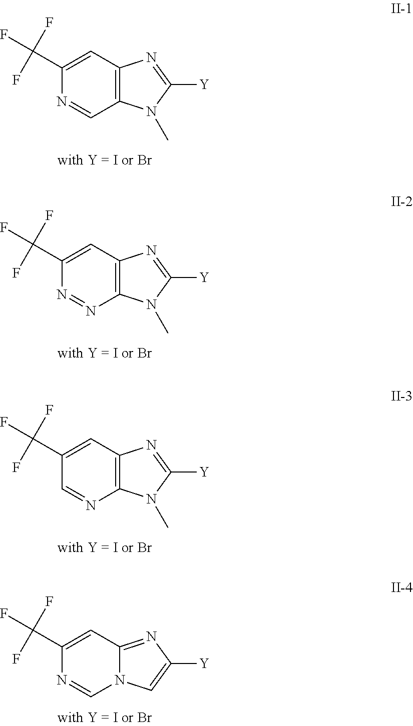

The present invention is elucidated in detail by the examples which follow, although the examples should not be interpreted in such a manner that they restrict the invention.

### EXAMPLE 1

### Synthesis of 3-methyl-6-(trifluoromethyl)-3H-imidazo[4,5-c]pyridine (Reactant)

N3-Methyl-6-(trifluoromethyl)pyridine-3,4-diamine (500 mg, 2.6 mmol), dissolved in formic acid (4 ml, 106 mmol), was heated with microwaves at 150° C. for 1 hour. After customary workup by addition of saturated aqueous ammonium chloride solution, the reaction mixture was extracted with ethyl acetate, and the combined organic phases were dried over Na₂SO₄ and concentrated in a membrane pump vacuum. After purification by column chromatography (ethyl acetate/cyclohexane), 3-methyl-6-(trifluoromethyl)-3H-imidazo[4,5-c]pyridine (480 mg, 91%) was obtained as a white solid. HPLC-MS: log P=1.09; Mass (m/z+1): 202.0; 1HNMR (D6-DMSO): δ 9.14 (s, 1H), 8.61 (s, 1H), 8.19 (s, 1H), 4.02 (s, 3H).

### EXAMPLE 2

### Synthesis of 2-iodo-3-methyl-6-(trifluoromethyl)-3H-imidazo[4,5-c]pyridine

To 3-methyl-6-(trifluoromethyl)-3H-imidazo[4,5-c]pyridine (201 mg, 1.0 mmol), dissolved in THF (1 ml), was added TMPZnCl.LiCl (1.31 M in THF, 0.82 ml, 1.1 mmol) at 25° C. under argon; this reaction solution was stirred for 10 min. Subsequently, iodine (508 mg, 2.0 mmol in 2 ml of THF) was added at 25° C. and the solution was stirred for a further 20 min. After customary workup by addition of saturated ammonium chloride and sodium thiosulphate solutions, the reaction mixture was extracted with ethyl acetate, and the combined organic phases were dried over Na₂SO₄ and concentrated in a membrane pump vacuum. After purification by column chromatography (ethyl acetate/cyclohexane), 2-iodo-3-methyl-6-(trifluoromethyl)-3H-imidazo[4,5-c]pyridine (301 mg, 93%) was obtained as a yellow solid. HPLC-MS: log P=1.77; Mass (m/z+1): 327.9; 1HNMR (D6-DMSO): δ 9.13 (s, 1H), 8.12 (s, 1H), 3.94 (s, 3H).

### EXAMPLE 3

### Synthesis of 2-bromo-3-methyl-6-(trifluoromethyl)-3H-imidazo[4,5-c]pyridine

To 3-methyl-6-(trifluoromethyl)-3H-imidazo[4,5-c]pyridine (201 mg, 1.0 mmol), dissolved in THF (0.8 ml), was added TMPZnCl.LiCl (1.35 M in THF, 0.82 ml, 1.1 mmol) at 25° C. under argon; this reaction solution was stirred for 10 min. Subsequently, bromine (224 mg, 1.4 mmol) was added at 0° C. and the solution was stirred for a further 20 min. After customary workup by addition of saturated ammonium chloride and sodium thiosulphate solutions, the reaction mixture was extracted with ethyl acetate, and the combined organic phases were dried over Na₂SO₄ and concentrated in a membrane pump vacuum. After purification by column chromatography (ethyl acetate/cyclohexane), 2-bromo-3-methyl-6-(trifluoromethyl)-3H-imidazo[4,5-c]pyridine (269 mg, 96%) was obtained as a yellow solid. HPLC-MS: log P=1.71; Mass (m/z+1): 281.0; 1HNMR (D6-DMSO): δ 9.15 (s, 1H), 8.17 (s, 1H), 3.96 (s, 3H).

### EXAMPLE 4

### Synthesis of 7-methyl-3-(trifluoromethyl)-7H-imidazo[4,5-c]pyridazine (Reactant)

N3-Methyl-6-(trifluoromethyl)pyridazine-3,4-diamine (1.0 g, 5.2 mmol), dissolved in formic acid (5 ml, 132 mmol), was heated with microwaves at 150° C. for 1 hour. After customary workup by addition of saturated aqueous ammonium chloride solution, the reaction mixture was extracted with ethyl acetate, and the combined organic phases were dried over Na₂SO₄ and concentrated in a membrane pump vacuum. After purification by column chromatography (ethyl acetate/cyclohexane), 7-methyl-3-(trifluoromethyl)-7H-imidazo[4,5-c]pyridazine (758 mg, 73%) was obtained as a white solid. HPLC-MS: log P=0.91; Mass (m/z+1): 203.1; 1HNMR (D6-DMSO): δ 8.92 (s, 1H), 8.62 (s, 1H), 4.08 (s, 3H).

### EXAMPLE 5

### Synthesis of 6-iodo-7-methyl-3-(trifluoromethyl)-7H-imidazo[4,5-c]pyridazine

To 7-methyl-3-(trifluoromethyl)-7H-imidazo[4,5-c]pyridazine (203 mg, 1.0 mmol), dissolved in THF (0.8 ml), was added TMPZnCl.LiCl (1.35 M in THF, 0.82 ml, 1.1 mmol) at 25° C. under argon; this reaction solution was stirred for 10 min. Subsequently, iodine (508 mg in 4 ml of THF) was added at 25° C. and the solution was stirred for a further 20 min. After customary workup by addition of saturated ammonium chloride and sodium thiosulphate solutions, the reaction mixture was extracted with ethyl acetate, and the combined organic phases were dried over Na₂SO₄ and concentrated in a membrane pump vacuum. After purification by column chromatography (ethyl acetate/cyclohexane), 6-iodo-7-methyl-3-(trifluoromethyl)-7H-imidazo[4,5-c]pyridazine (230 mg, 70%) was obtained as a yellow solid. HPLC-MS: log P=1.66; Mass (m/z+1): 329.0; 1HNMR (D6-DMSO): δ 8.53 (s, 1H), 3.99 (s, 3H).

### EXAMPLE 6

### Synthesis of 3-methyl-6-(trifluoromethyl)-3H-imidazo[4,5-b]pyridine (Reactant)

N2-Methyl-5-(trifluoromethyl)pyridine-2,3-diamine (500 mg, 2.61 mmol), dissolved in formic acid (4 ml, 106 mmol), was heated with microwaves at 150° C. for 1 hour. After customary workup by addition of saturated aqueous ammonium chloride solution, the reaction mixture was extracted with ethyl acetate, and the combined organic phases were dried over Na₂SO₄ and concentrated in a membrane pump vacuum. After purification by column chromatography (ethyl acetate/cyclohexane), 3-methyl-6-(trifluoromethyl)-3H-imidazo[4,5-c]pyridine (385 mg, 74%) was obtained as a white solid. HPLC-MS: log P=1.44; Mass (m/z+1): 202.1; 1HNMR (D6-DMSO): δ 8.77 (s, 1H), 8.67 (s, 1H), 8.52 (s, 1H), 3.90 (s, 3H).

### EXAMPLE 7

### Synthesis of 2-iodo-3-methyl-6-(trifluoromethyl)-3H-imidazo[4,5-b]pyridine

To 3-methyl-6-(trifluoromethyl)-3H-imidazo[4,5-b]pyridine (100 mg, 0.49 mmol), dissolved in THF (0.4 ml), was added TMPZnCl.LiCl (1.35 M in THF, 0.41 ml, 0.54 mmol) at 25° C. under argon; this reaction solution was stirred for 10 min. Subsequently, iodine (252 mg in 1 ml of THF) was added at 25° C. and the solution was stirred for a further 20 min. After customary workup by addition of saturated ammonium chloride and sodium thiosulphate solutions, the reaction mixture was extracted with ethyl acetate, and the combined organic phases were dried over Na₂SO₄ and concentrated in a membrane pump vacuum. After purification by column chromatography (ethyl acetate/cyclohexane), 2-iodo-3-methyl-6-(trifluoromethyl)-3H-imidazo[4,5-b]pyridine (111 mg, 69%) was obtained as a yellow solid. HPLC-MS: log P=2.29; Mass (m/z+1): 328.0; 1HNMR (D6-DMSO): δ 8.71 (s, 1H), 8.47 (s, 1H), 3.82 (s, 3H).

1. Process for preparing compound of formula (II)
Q-Hal (II)
in which
Q is a structural element 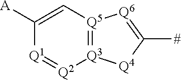
where the symbol # indicates the bond to the rest of the molecule and
Q¹ is N or CR⁶,
Q² is N or CR⁶,
Q³ is N or C,
Q⁴ is O, S, N or NR⁷,
Q⁵ is N or C,
Q⁶ is N or CH,
R⁶ is hydrogen, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
R⁷ is (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo (C₃-C₆)cycloalkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl, and
A is hydrogen, cyano, halogen, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, (C₁-C₄)alkoxy, (C₁-C₄)haloalkoxy, (C₁-C₄)alkoxyimino, (C₁-C₄)alkyl thio, (C₁-C₄)haloalkylthio, (C₁-C₄)alkylsulphinyl, (C₁-C₄)haloalkylsulphinyl, (C₁-C₄)alkylsulphonyl, (C₁-C₄)haloalkylsulphonyl, (C₁-C₄)alkylsulphonyloxy, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, aminosulphonyl, (C₁-C₄)alkylaminosulphonyl or di-(C₁-C₄)alkylaminosulphonyl,
or A is —O—CF₂—O— and, together with Q¹ and the carbon atom to which it is bonded, forms a five-membered ring where Q¹ is carbon, and
Hal is halogen,
comprising a) reacting, a compound Q-H in which Q is as defined above
with an organozinc base of the structure (NR³R⁴)—Zn—R² or (NR³R⁴)₂—Zn in which
R² is halogen or —O-pivaloyl and
R³ and R⁴ together form a —(CH₂)₄—, —(CH₂)₅— or —(CH₂)₂O(CH₂)₂— group, where each of these groups may optionally be substituted by 1, 2, 3 or 4 R⁵ radicals and R⁵ is selected from the group consisting of methyl, ethyl, n-propyl and i-propyl,
to give a compound of formula (IIIa) or formula (IIIb) 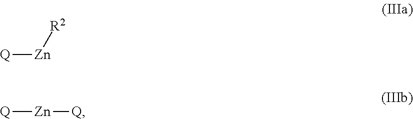
in which Q and R² each have the definitions given above,
and b) reacting said compound of formula (IIIa) or (IIIb) with a compound of the structure X-Hal in which X is halogen and Hal has the abovementioned definition to give the compound of formula (II).

2. Process according to claim 1, wherein
Q is a structural element from the group of Q1 to Q15 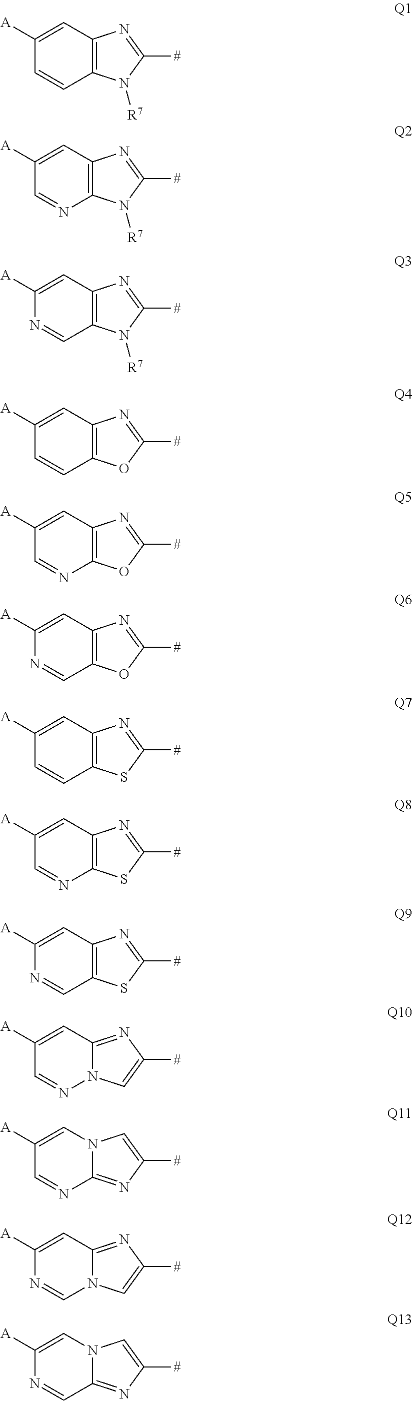 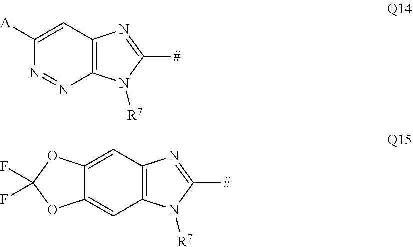
R⁷ is (C₁-C₄)alkyl, (C₁-C₄)haloalkyl, (C₁-C₄)alkoxy-(C₁-C₄)alkyl, (C₁-C₄)haloalkoxy-(C₁-C₄)alkyl, (C₁-C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
A is fluorine, chlorine, bromine, fluoromethyl, difluoromethyl, trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethoxy, difluorochloromethoxy, dichlorofluoromethoxy, trifluoromethylthio, trifluoromethylsulphinyl or trifluoromethylsulphonyl,
Hal and X have the same definition and are fluorine, chlorine, iodine or bromine, and
R² is halogen, optionally chlorine, bromine or iodine.

3. Process according to claim 2, wherein
Q is a structural element from the group of Q2, Q3, Q10, Q12, Q14 or Q15,
R⁷ is (C₁-C₄)alkyl or (C₁-C₄)alkoxy-(C₁-C₄)alkyl,
A is trifluoromethyl, fluoroethyl (CH₂CFH₂, CHFCH₃), difluoroethyl (CF₂CH₃, CH₂CHF₂, CHFCFH₂), trifluoroethyl, (CH₂CF₃, CHFCHF₂, CF₂CFH₂), tetrafluoroethyl (CHFCF₃, CF₂CHF₂), pentafluoroethyl, trifluoromethylthio, trifluoromethylsulphinyl or trifluoromethylsulphonyl,
Hal and X have the same definition and are iodine or bromine, and
R² is chlorine.

4. Process according to claim 1, wherein
Q is the structural element Q2, Q3, Q12 or Q14,
R⁷ is methyl, ethyl, n-propyl or isopropyl, especially methyl,
A is trifluoromethyl,
Hal and X have the same definition and are iodine or bromine, and
R² is chlorine.

5. Process according to claim 1, wherein
R³ and R⁴ together form a —(CH₂)₅— group substituted by 4 methyl groups.

6. Process according to claim 1, wherein the organozinc base is a compound of formula (V)
(TMP)ₓZnCl₂₋ₓ, (V)
in which x is the number 1 or 2.

7. Process according to claim 1, wherein the organozinc base is present in conjunction with an alkali metal halide or alkaline earth metal halide, preferably lithium chloride or magnesium chloride.

8. Process according to claim 1, wherein the organozinc base is used in a total amount of 0.5 to 5 equivalents, based on the compound Q-H.

9. Process according to claim 1, wherein the compound X-Hal is an elemental halogen, optionally F₂, Cl₂, Br₂ or I₂.

10. Process according to claim 1, wherein the compound X-Hal is used in a total amount of 0.5 to 10.0 equivalents, based on the compound Q-H.

11. Process according to claim 1, which is conducted in the presence of a solvent selected from the group consisting of tetrahydrofuran (THF), 1,4-dioxane, diethyl ether, diglyme, methyl tert-butyl ether (MTBE), tert-amyl methyl ether (TAME), 2-methyl-THF, toluene, xylenes, mesitylene, ethylene carbonate, propylene carbonate, N,N-dimethylacetamide, N,N-dimethylformamide (DMF), N-methylpyrrolidone (NMP), N-ethyl-2-pyrrolidone (NEP), N-butyl-2-pyrrolidone (NBP); N,N′-dimethylpropyleneurea (DMPU), halohydrocarbon, aromatic hydrocarbon, chlorohydrocarbon, tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, 1,2-dichlorobenzene, chlorotoluene, trichlorobenzene; 4-methoxybenzene, fluorinated aliphatic, fluorinated aromatic, trichlorotrifluoroethane, benzotrifluoride and 4-chlorobenzotrifluoride, or a mixture of at least two of these solvents with one another.

12. Process according to claim 11, wherein the solvent is THF or N,N-dimethylformamide (DMF).

13. Process according to claim 1, wherein a) is conducted at a temperature between 0° C. and 80° C.

14. Process according to claim 1, wherein b) is conducted at a temperature between 0° C. and 40° C.

15. Compound of the structure Q-H, selected from the following compounds:

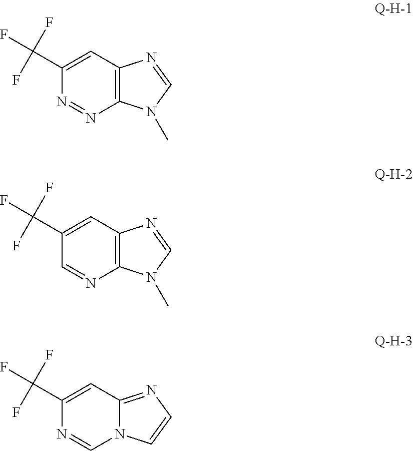

16. Compound of formula (IIIa), selected from the following compounds:

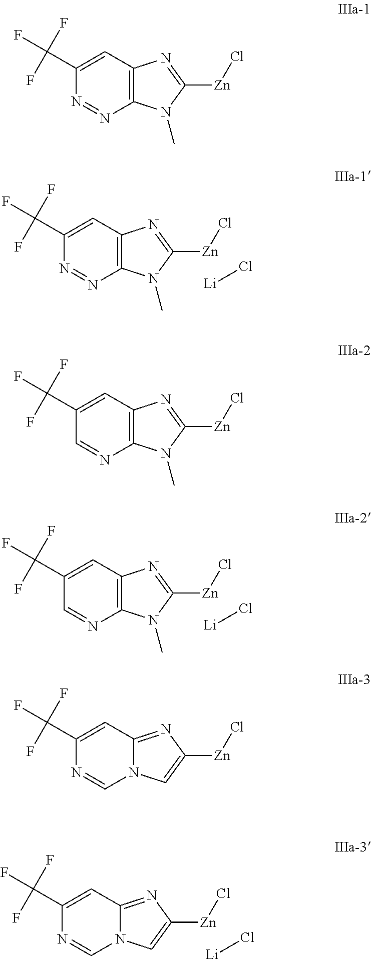

17. Compound of formula (II)
Q-Hal (II)
in which
Q is a structural element 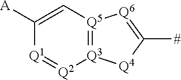
where the symbol # indicates the bond to the rest of the molecule and
Q¹ is N or CR⁶,
Q² is N or CR⁶,
Q³ is N or C,
Q⁴ is O, S, N or NR⁷,
Q⁵ is N or C,
Q⁶ is N or CH,
R⁶ is hydrogen, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, halo(C₃-C₆)cycloalkyl, C₄)alkylthio-(C₁-C₄)alkyl, (C₁-C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
R⁷ is (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₄)haloalkynyl, (C₃-C₆)cycloalkyl-(C₃-C₆)cycloalkyl, (C₁-C₄)alkyl-(C₃-C₆)cycloalkyl, halo(C₃-C₆)cycloalkyl, (C₁-C₄)alkylthio-(C₁-C₄)alkyl, C₄)alkylsulphinyl-(C₁-C₄)alkyl, (C₁-C₄)alkylsulphonyl-(C₁-C₄)alkyl or (C₁-C₄)alkylcarbonyl-(C₁-C₄)alkyl,
A is hydrogen, cyano, halogen, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₃-C₆)cycloalkyl, (C₃-C₆)cycloalkyl-(C₃-C₆)cycloalkyl, (C₁-C₄)alkyl-(C₃-C₆)cycloalkyl, (C₁-C₄)alkoxy, (C₁-C₄)haloalkoxy, (C₁-C₄)alkoxyimino, (C₁-C₄)alkylthio, (C₁-C₄)haloalkylthio, (C₁-C₄)alkylsulphinyl, (C₁-C₄)haloalkylsulphinyl, (C₁-C₄)alkylsulphonyl, (C₁-C₄)haloalkylsulphonyl, (C₁-C₄)alkylsulphonyloxy, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, aminosulphonyl, (C₁-C₄)alkylaminosulphonyl or di-(C₁-C₄)alkylaminosulphonyl,
or A is —O—CF₂—O— and, together with Q¹ and the carbon atom to which it is bonded, forms a five-membered ring where Q¹ is carbon, and
Hal is fluorine, iodine or bromine, optionally iodine or bromine.
